(19) United States
(12) Patent Application Publication
(10) Pub. No.: US 2026/0191694 A1
(43) Pub. Date: Jul. 9, 2026

(54) WOUND DRESSING AND WOUND THERAPY SYSTEM

(71) Applicant: Solventum Intellectual Properties Company, Maplewood, MN (US)

(72) Inventor: Pedro MALDONADO, Saint Paul, MN (US)

(21) Appl. No.: 19/132,092

(22) PCT Filed: Dec. 4, 2023
(86) PCT No.: PCT/IB2023/062206

§ 371 Date: May 22, 2025

Related U.S. Application Data
(60) Provisional application No. 63/431,047, filed on Dec. 8, 2022.

(51) Int. Cl. A61F 13/05; A61F 13/0203; A61M 1/00

(52) U.S. Cl. CPC A61F 13/05; A61F 13/0203; A61M 1/915

(57) ABSTRACT

A wound dressing for a wound is disclosed. The wound dressing includes an absorbent pad. The wound dressing further includes a cover layer attached to the absorbent pad. The cover layer further includes at least a portion of a channel wall. The wound dressing further includes a channel defined by the channel wall. The channel surrounds the absorbent pad and is spaced apart from the absorbent pad. The channel is continuous and fluidly isolated from the absorbent pad. The channel is configured to receive a negative pressure. The wound dressing further includes a manifold member disposed within the channel. The manifold member at least partially engages the channel wall. The manifold member is collapsible in response to the negative pressure applied to the channel.

### CROSS-REFERENCE TO RELATED APPLICATIONS

This application is a U.S. National Stage Entry of PCT International Application No. PCT/IB 2023/062206, filed Dec. 4, 2023, which claims the benefit of priority to U.S. Provisional Application No. 63/431,047, filed on Dec. 8, 2022, each of which are incorporated herein by reference in their entirety.

### TECHNICAL FIELD

The present disclosure relates generally to a wound dressing and a wound therapy system for a wound.

### BACKGROUND

Negative pressure wound therapy (NPWT) involves applying a negative pressure to a wound site to promote wound healing. NPWT may be used to heal wounds through various mechanisms of action (MOA), ranging from removal of infectious material and wound exudate to reducing edema for improved perfusion.

However, application of the negative pressure to the wound site by way of a foam that engages the wound may cause atmospheric pressure to be transferred to the wound, which may cause compression, thereby decreasing perfusion below the foam/wound interface. Therefore, there is a need for a wound dressing that improves both the healing potential of the wound using NPWT and perfusion at the wound site.

### SUMMARY

In a first aspect, the present disclosure provides a wound dressing for a wound. The wound dressing includes an absorbent pad including a first pad surface and a second pad surface opposite to the first pad surface. The second pad surface is configured to face the wound. The wound dressing further includes a cover layer including a first cover surface and a second cover surface opposite to the first cover surface. The second cover surface is attached to and faces the first pad surface. The cover layer includes at least a portion of a channel wall. The wound dressing further includes a channel defined by the channel wall. The channel surrounds the absorbent pad and is spaced apart from the absorbent pad. The channel is continuous, and fluidly isolated from the absorbent pad. The channel is configured to receive a negative pressure. The wound dressing further includes a manifold member disposed within the channel and at least partially engaging the channel wall. The manifold member is collapsible in response to the negative pressure applied to the channel.

In a second aspect, the present disclosure provides a wound therapy system for a wound. The wound therapy system includes a wound dressing. The wound dressing includes an absorbent pad including a first pad surface and a second pad surface opposite to the first pad surface. The second pad surface is configured to face the wound. The wound dressing further includes a cover layer including a first cover surface and a second cover surface opposite to the first cover surface. The second cover surface is attached to and faces the first pad surface. The cover layer includes at least a portion of a channel wall. The wound dressing further includes a channel defined by the channel wall. The channel surrounds the absorbent pad and is spaced apart from the absorbent pad. The channel is continuous and fluidly isolated from the absorbent pad. The wound dressing further includes a manifold member disposed within the channel and at least partially engaging the channel wall. The wound dressing further includes a negative pressure source fluidly communicating with the channel and configured to apply a negative pressure to the channel. The manifold member collapses in response to the negative pressure applied to the channel.

The details of one or more examples of the disclosure are set forth in the accompanying drawings and the description below. Other features, objects, and advantages of the disclosure will be apparent from the description and drawings, and from the claims.

### BRIEF DESCRIPTION OF THE DRAWINGS

Exemplary embodiments disclosed herein may be more completely understood in consideration of the following detailed description in connection with the following figures. The figures are not necessarily drawn to scale. In particular, thicknesses of certain layers in proportion to certain other items are exaggerated for ease of illustration and clarity purposes. Like numbers used in the figures refer to like components. However, it will be understood that the use of a number to refer to a component in a given figure is not intended to limit the component in another figure labeled with the same number.

FIG. 1 is a schematic plan view of a wound therapy system according to an embodiment of the present disclosure;

FIG. 2A is a schematic cross-sectional view of a wound dressing taken along a line 1-1 of FIG. 1 according to an embodiment of the present disclosure;

FIG. 2B is a schematic zoomed-in view of a portion of the wound dressing of FIG. 2A according to an embodiment of the present disclosure;

FIGS. 3A-3E are schematic cross-sectional views of a manifold member of the wound dressing according to some embodiments of the present disclosure; and

FIG. 4 is a schematic cross-sectional view of a wound dressing according to another embodiment of the present disclosure.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

In the following disclosure, the following definitions are adopted.

As recited herein, all numbers should be considered modified by the term “about”. As used herein, “a,” “an,” “the,” “at least one,” and “one or more” are used interchangeably.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20 % for quantifiable properties).

The term “substantially”, unless otherwise specifically defined, means to a high degree of approximation (e.g., within +/−10% for quantifiable properties) but again without requiring absolute precision or a perfect match.

The term “about”, unless otherwise specifically defined, means to a high degree of approximation (e.g., within +/−5% for quantifiable properties) but again without requiring absolute precision or a perfect match.

Terms such as same, equal, uniform, constant, strictly, and the like, are understood to be within the usual tolerances or measuring error applicable to the particular circumstance rather than requiring absolute precision or a perfect match.

As used herein, the terms “first” and “second” are used as identifiers. Therefore, such terms should not be construed as limiting of this disclosure. The terms “first” and “second” when used in conjunction with a feature or an element can be interchanged throughout the embodiments of this disclosure.

As used herein, when a first material is termed as “similar” to a second material, at least 90 weight % of the first and second materials are identical and any variation between the first and second materials comprises less than about 10 weight % of each of the first and second materials.

As used herein, “at least one of A and B” should be understood to mean “only A, only B, or both A and B”.

Unless specified or limited otherwise, the terms “attached,” “connected,” “coupled,” and variations thereof, are used broadly and encompass both direct and indirect attachments, connections, and couplings.

As used herein, the term “moisture vapor transmission rate” or “MVTR” refers to the permissible moisture volume from one side of the substrate web to the other side of the substrate web per area unit (e.g., per square meter) and per time unit (e.g., per one day).

As used herein, the term “configured to” is at least as restrictive as the term “adapted to” and requires actual design intention to perform the specified function rather than mere physical capability of performing such a function.

As used herein, the term “layer” refers to a thickness of a material or blend of materials. Layers may be continuous or discontinuous.

As used herein, the term “adhesive layer” refers to a layer of adhesive material disposed on one or more layers to promote an adhesion of the one or more layers to each other or to another surface. Adhesive layers may be patterned.

As used herein, the term “perimeter” refers to a border, a circumference, or an outer boundary of a layer. It may be noted that a perimeter may be of any shape, such as, but not limited to, triangular, rectangular, octagonal, square, circular, oval, trapezoidal, pentagonal, hexagonal, and so forth.

As used herein, the term “wounds” may include, for example, chronic, acute, traumatic, subacute, closed surgical wounds or dehiscence wounds, partially thick burns, ulcers (such as, diabetic, compressive, or venous insufficiency ulcers), flaps, and grafts. The wound may also include an open abdomen area of a patient.

As used herein, the term “wound site” may include a tissue site, such as, bone tissue, adipose tissue, muscle tissue, nerve tissue, skin tissue, vascular tissue, connective tissue, cartilage, tendons, or ligaments. The term “wound site” may also refer to an area of a tissue that is not necessarily a wound or a defect but may be desired to add or promote additional tissue growth. For example, negative pressure therapy can be used in a particular tissue area to grow additional tissue that can be harvested or transplanted to another tissue site. The wound site may also include an area wherein a surgical incision has been previously performed.

The present disclosure relates to a wound dressing for a wound. The wound dressing includes an absorbent pad including a first pad surface and a second pad surface opposite to the first pad surface. The second pad surface is configured to face the wound. The wound dressing further includes a cover layer including a first cover surface and a second cover surface opposite to the first cover surface. The second cover surface is attached to and faces the first pad surface. The cover layer includes at least a portion of a channel wall. The wound dressing further includes a channel defined by the channel wall. The channel surrounds the absorbent pad and is spaced apart from the absorbent pad. The channel is continuous and fluidly isolated from the absorbent pad. The channel is configured to receive a negative pressure. The wound dressing further includes a manifold member disposed within the channel and at least partially engaging the channel wall. The manifold member is collapsible in response to the negative pressure applied to the channel.

The wound dressing of the present disclosure may promote perfusion at a wound site and promote healing of the wound.

Upon application of the negative pressure to the channel, the manifold member may collapse (substantially along an in-plane axis of the wound dressing), which may cause a skin around the wound (or periwound) to stretch. This may dilate vessels/lymphatics and allow improved fluid movement to the wound. The manifold member may define a cross-sectional plane, a width axis disposed in the cross-sectional plane along a width of the manifold member, and a height axis perpendicular to the width axis. In some examples, the manifold member may collapse substantially along the width axis of the manifold member upon application of the negative pressure to the channel, thereby increasing the effect of vessel dilation.

In some examples, application of the negative pressure to the channel may further result in application of the negative pressure to the periwound, which contains critical vasculature that supplies the wound with elements needed for healing, such as improved perfusion, oxygenation, and stress-induced cell proliferation.

Moreover, as the negative pressure may only be applied to the channel, the negative pressure source of a cheaper and less powerful variety may be used with the wound dressing as compared to conventional negative pressure systems. Advantageously, the channel may additionally function as a protective border and absorb loads from accidental bumps, thereby providing protection to the wound.

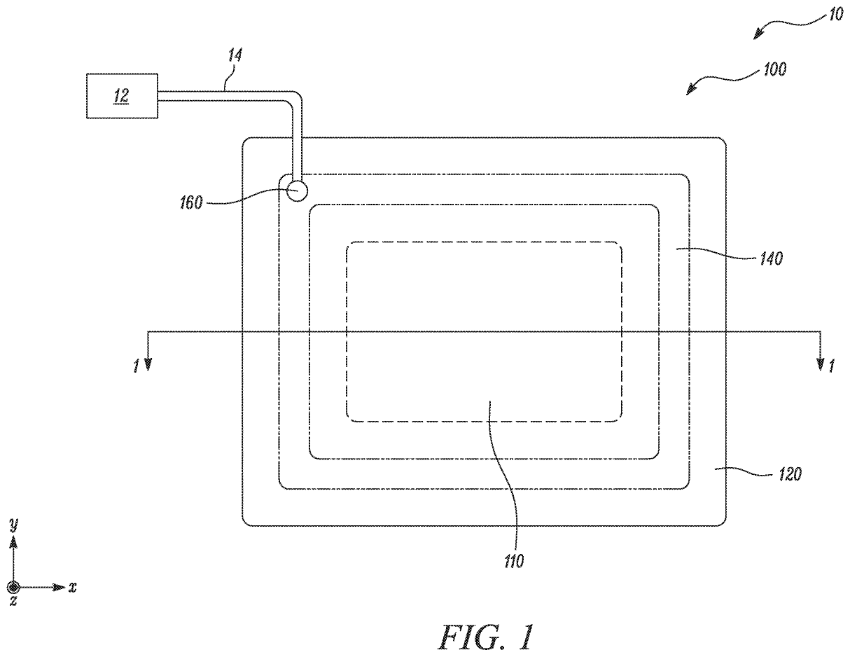

Referring now to the Figures, FIG. 1 illustrates a schematic plan view of a wound therapy system 10 for a wound according to an embodiment of the present disclosure.

The wound therapy system 10 includes a negative pressure source 12 (schematically depicted by a block in FIG. 1) and a wound dressing 100. The negative pressure source 12 may be fluidly coupled to a portion of the wound dressing 100.

The negative pressure source 12 may include any source of reduced pressure, such as a vacuum pump, a suction pump, a wall suction port (available at many healthcare facilities), a micro-pump, and the like, which can be operated to apply a negative pressure or reduced pressure to the portion of the wound dressing 100 that is fluidly coupled thereto.

“Negative pressure” generally refers to a pressure less than a local ambient pressure, such as the ambient pressure in a local environment external to a sealed therapeutic environment. In many cases, the local ambient pressure may also be the atmospheric pressure at which a tissue or wound site is located. Alternatively, the pressure may be less than a hydrostatic pressure associated with tissue at a tissue or wound site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative pressure source 12 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

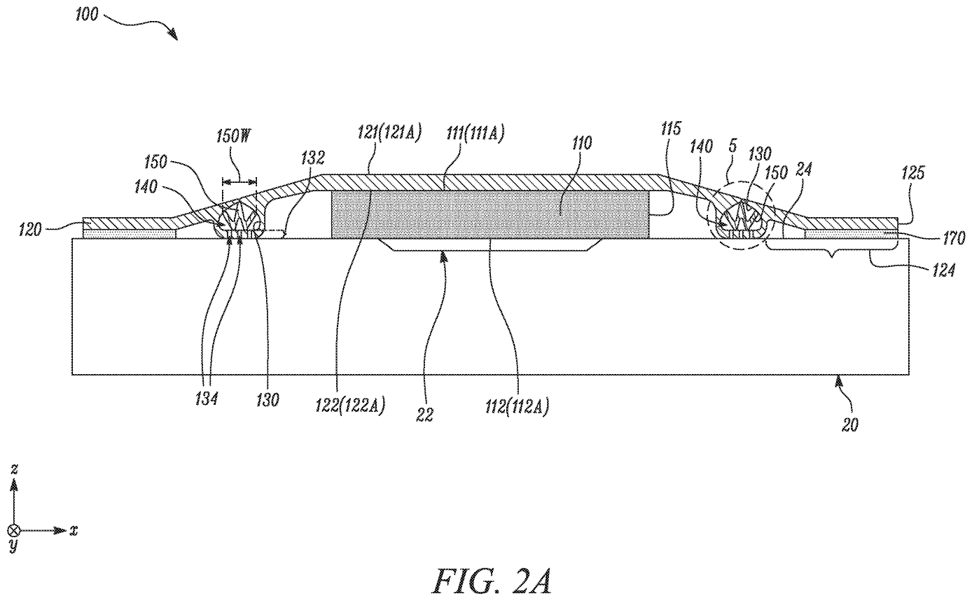

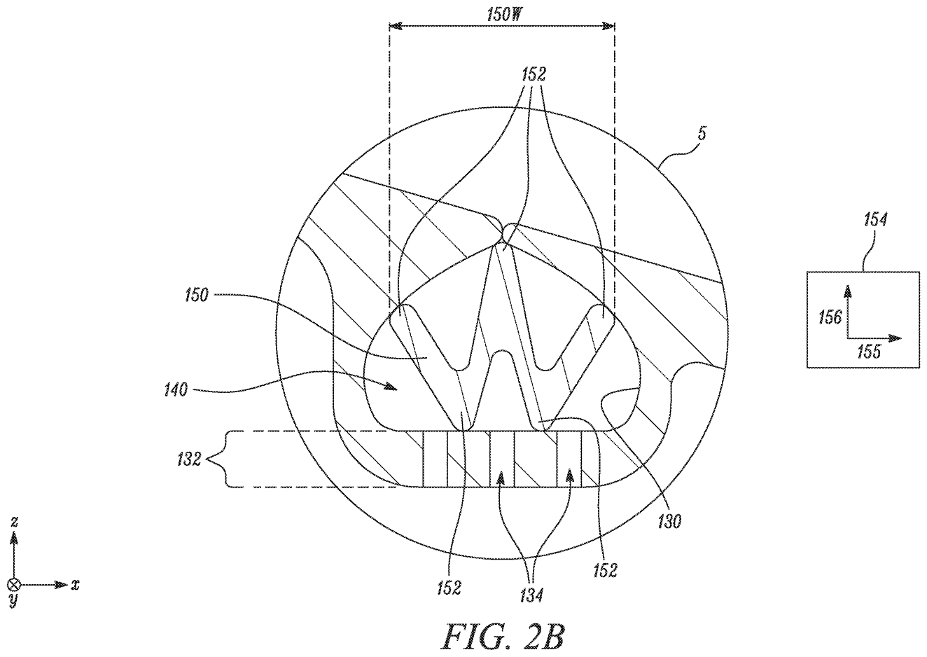

FIG. 2A illustrates a schematic cross-sectional view of the wound dressing 100 taken along a line 1-1 of FIG. 1 according to an embodiment of the present disclosure. FIG. 2A further illustrates a wound site 20 including a wound 22 and a skin 24 around the wound 22. A region around the wound 22 may be referred to as “periwound.” In FIG. 2A, the wound dressing 100 is removably connected to the skin 24 or the periwound.

Referring to FIGS. 1 and 2A, the wound dressing 100 defines mutually orthogonal x, y, and z-axes. The x-axis is defined along a length of the wound dressing 100, while the y-axis is defined along a breadth of the wound dressing 100. The z-axis is defined along a thickness of the wound dressing 100.

The wound dressing 100 includes an absorbent pad 110. The absorbent pad 110 includes a first pad surface 111 and a second pad surface 112 opposite to the first pad surface 111. The second pad surface 112 is configured to face the wound 22. In the illustrated embodiment of FIG. 2A, the second pad surface 112 directly engages with the wound 22.

The absorbent pad 110 may be configured to absorb and remove a wound exudate from the wound 22. The absorbent pad 110 may include a foam. For example, the absorbent pad 110 may include a polyurethane foam or a polyethylene foam. In some embodiments, the absorbent pad 110 may be hydrophilic. The absorbent pad 110 may draw the wound exudate and other fluids away from the wound 22 by capillary flow or other wicking mechanisms.

The first pad surface 111 of the absorbent pad 110 may include an area 111A. The second pad surface 112 of the absorbent pad 110 may include an area 112A. The area 112A of the second pad surface 112 may be greater than an area of the wound 22. The absorbent pad 110 may further include a pad perimeter 115 extending between the first pad surface 111 and the second pad surface 112. The pad perimeter 115 may surround the wound 22. The absorbent pad 110 may fully cover the wound 22.

The wound dressing 100 further includes a cover layer 120. The cover layer 120 includes a first cover surface 121 and a second cover surface 122 opposite to the first cover surface 121. The second cover surface 121 is attached to and faces the first pad surface 111. The second cover surface 121 may be attached to the first pad surface 111 by any suitable method, such as adhesive lamination, heat lamination, etc.

The cover layer 120 may further include a cover perimeter 125 extending between the first cover surface 121 and the second cover surface 122. The first cover surface 121 of the cover layer 120 may include an area 121A. The second cover surface 122 of the cover layer 120 may include an area 122A. The area 122A of the second cover surface 122 may be greater than the area 111A of the first pad surface 111, such that the cover perimeter 125 surrounds the absorbent pad 110.

The cover layer 120 may provide the wound 22 with a bacterial barrier and protect the wound 22 from physical trauma. The cover layer 120 may be constructed from a material that can reduce evaporative losses and provide a fluid seal between two components or two environments, such as between a therapeutic environment and a local external environment. The cover layer 120 may include, for example, an elastomeric film or a membrane that can provide a seal adequate to maintain a negative pressure. In some applications, the cover layer 120 may have a high moisture-vapor transmission rate (MVTR). For example, the MVTR may be at least 250 grams per square meter per twenty-four hours, measured using an upright cup technique according to ASTM E 96/E96M Upright Cup Method at 38° C. and 10% relative humidity (RH). In some embodiments, an MVTR up to 5,000 grams per square meter per twenty-four hours may provide effective breathability and mechanical properties.

In some embodiments, the cover layer 120 may be a non-porous polymer drape or film, such as a polyurethane film that is permeable to water vapor but impermeable to liquid. Such drapes typically have a thickness (along the z-axis) in the range of 25 microns to 50 microns. For permeable materials, the permeability may be low enough that a desired negative pressure may be maintained. The cover layer 120 may include, for example, one or more of the following materials:
polyurethane (PU), such as hydrophilic polyurethane; cellulosics; hydrophilic polyamides;
polyvinyl alcohol; polyvinyl pyrrolidone; hydrophilic acrylics; silicones, such as hydrophilic silicone elastomers; natural rubbers; polyisoprene; styrene butadiene rubber; chloroprene rubber;
polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polyamide copolymers. Such materials are commercially available as, for example, Tegaderm® drape, commercially available from 3M Company, Minneapolis Minn. ; polyurethane (PU) drape, commercially available from Avery Dennison Corporation, Pasadena, Calif. ; polyether block polyamide copolymer (PEBAX), for example, from Arkema S. A., Colombes, France; and Inspire 2301 and Inpsire 2327 polyurethane films, commercially available from Coveris Advanced Coatings, Wrexham, United Kingdom.

The cover layer 120 further includes at least a portion of a channel wall 130. In the illustrated embodiment of FIG. 2A, the cover layer 120 is sealed to itself to form the channel wall 130. That is, in the illustrated embodiment of FIG. 2A, the cover layer 120 entirely forms the channel wall 130.

The wound dressing 100 further includes a channel 140 defined by the channel wall 130. The channel 140 surrounds the absorbent pad 110 and is spaced apart from the absorbent pad 110. Further, the channel 140 is continuous and fluidly isolated from the absorbent pad 110.

The channel 140 is configured to receive a negative pressure. As shown in FIG. 1, the negative pressure source 12 may fluidly communicate with the channel 140. The negative pressure source 12 may be fluidly coupled to the channel 140. The negative pressure source 12 may be fluidly coupled to the channel 140 by a tube 14. The negative pressure source 12 may be configured to apply the negative pressure to the channel 140. The wound dressing 100 may further include a port 160 fluidly coupled to the channel 140. Specifically, the port 160 may be fluidly coupled to the channel 140 and the negative pressure source 12. The channel 140 may be configured to receive the negative pressure via the port 160. Specifically, the channel 140 may be configured to receive the negative pressure from the negative pressure source 12 via the port 160. In some embodiments, the cover layer 120 includes the port 160 extending therethrough and fluidly communicating with the channel 140.

The negative pressure source 12 may be fluidly coupled to only the channel 140 (e.g., via the port 160) of the wound dressing 100, such that the negative pressure source 12 is fluidly disconnected from the absorbent pad 110. Therefore, only the channel 140 of the wound dressing 100 may receive the negative pressure, such that the absorbent pad 110 does not receive any negative pressure.

The channel wall 130 may include a bottom portion 132 configured to face the skin 24 around the wound 22. The bottom portion 132 may include a plurality of through openings 134. In other words, the bottom portion 132 of the channel wall 130 may be fenestrated. Therefore, the negative pressure applied to the channel 140 may be further applied to the skin 24 around the wound 22 via the plurality of through openings 134. In some embodiments, the bottom portion 132 further includes an adhesive configured to adhere the bottom portion 132 to the skin 24. In some embodiments, the bottom portion 132 includes both the adhesive and the plurality of through openings 134.

The wound dressing 100 further includes a manifold member 150 disposed within the channel 140. The manifold member 150 at least partially engages the channel wall 130. The manifold member 150 is collapsible in response to the negative pressure applied to the channel 140. That is, the manifold member 150 may collapse in response to the negative pressure applied to the channel 140.

FIG. 2B illustrates a schematic zoomed-in view of a portion 5 of the wound dressing 100 of FIG. 2A according to an embodiment of the present disclosure. The x, y, and z-axes of the wound dressing 100 are also shown in FIG. 2B.

The manifold member 150 may include a cross-sectional plane 154 (schematically depicted by a rectangle in FIG. 2B). In the illustrated view of FIG. 2B, the cross-sectional plane 154 is the z-x plane of the wound dressing 100. Alternatively, the cross-sectional plane 154 may be the y-z plane of the wound dressing 100. The cross-sectional plane 154 may include the z-axis of the wound dressing 100 and the x or the y-axis.

The manifold member 150 may further include a width axis 155 disposed in the cross-sectional plane 154 along a width of the manifold member 150. The width axis 155 may be aligned with the x-axis or the y-axis of the wound dressing 100. The manifold member 150 may further include a height axis 156 perpendicular to the width axis 155. The height axis 156 may be aligned with the z-axis of the wound dressing 100. The manifold member 150 may be collapsible substantially along the width axis 155 upon application of the negative pressure to the channel 140.

Referring to FIGS. 1-2B, the channel 140 may extend along both the x and y-axes to surround the absorbent pad 110. In some embodiments, in a portion of the channel 140 extending substantially along the x-axis, the cross-sectional plane 154 may be aligned with the y-z plane, such that the manifold member 150 may be collapsible substantially along the y-axis upon application of the negative pressure to the channel 140. Further, in some embodiments, in a portion of the channel 140 extending substantially along the y-axis, the cross-sectional plane 154 may be aligned with the z-x plane, such that the manifold member 150 may be collapsible substantially along the x-axis upon application of the negative pressure to the channel 140.

The manifold member 150 may include a maximum width 150W (also shown in FIG. 2B) along the width axis 155 (or the x-axis in the depicted views of FIGS. 2A and 2B). The maximum width 150W of the manifold member 150 may decrease upon application of the negative pressure to the channel 140. In some embodiments, the maximum width 150W upon application of the negative pressure to the channel 140 may be in the range of 0.5 to 0.9 times of the maximum width 150W in the absence of the negative pressure to the channel 140.

The manifold member 150 may be made from any suitable material having desired collapsibility. In some embodiments, the manifold member 150 may include foam. The foam may be porous and collapsible (in the x and y axes). For example, the manifold member 150 may be an open-cell, reticulated polyurethane foam such as GranuFoam® dressing available from Kinetic Concepts, Inc. of San Antonio, Tex.

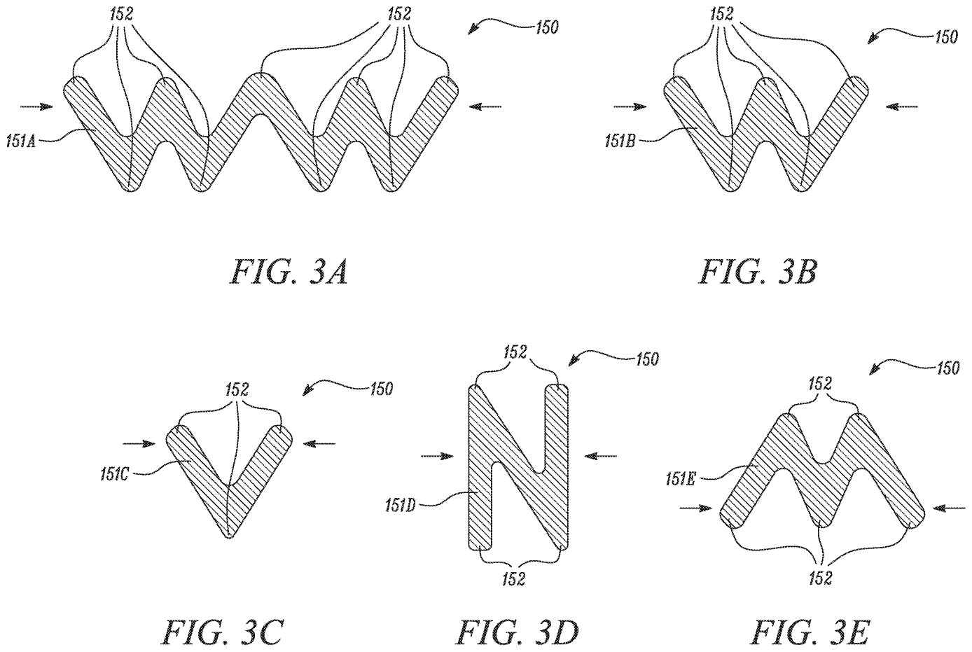

As discussed above, the manifold member 150 at least partially engages the channel wall 130. Specifically, the manifold member 150 may include a plurality of vertices 152. Each of the plurality of vertices 152 may engage the channel wall 130. The plurality of vertices 152 is also shown in FIGS. 3A-3E.

FIGS. 3A-3E illustrate different cross-sectional shapes of the manifold member 150 according to some embodiments of the present disclosure. In FIGS. 3A-3F, a direction of collapsibility of the manifold member 150 is indicated by opposing arrows.

As shown in FIG. 3A, in some embodiments, the manifold member 150 may include a zig-zag shape 151A. As shown in FIG. 3B, in some embodiments, the manifold member 150 may include a W-shape 151B. As shown in FIG. 3C, in some embodiments, the manifold member 150 may include a V-shape 151C. As shown in FIG. 3D, in some embodiments, the manifold member 150 may include an N-shape 151D. As shown in FIG. 3E, in some embodiments, the manifold member 150 may include an M-shape 151E. Specifically, in some embodiments, the manifold member 150 may include the zig-zag shape 151A, the W-shape 151B, the V-shape 151C, or the N-shape 151D. However, it may be noted that the manifold member 150 may include any suitable shape based on desired application attributes.

Referring to FIGS. 1 and 2A, the wound dressing 100 may be removably connected to the skin 24 by any suitable method. For example, the wound dressing 100 may be removably connected to the skin 24 by an adhesive. Alternatively, the wound dressing 100 may be removably connected to the skin 24 by adhesive tapes, and the like.

As shown in FIG. 2A, the second cover surface 122 of the cover layer 120 may include an outer portion 124 disposed between the cover perimeter 125 and the channel 140. In some embodiments, the wound dressing 100 may further include an adhesive layer 170 at least partially disposed on the outer portion 124. The adhesive layer 170 may be configured to removably connect the wound dressing 100 to the skin 24 around the wound 22. The adhesive layer 170 may include any suitable medically acceptable adhesive. For example, the adhesive layer 170 may include a pressure sensitive adhesive (PSA). The PSA may be based on acrylate ester copolymers, polyvinyl ethyl ether, and/or polyurethane. The PSA may include an elastomer dissolved or dispersed in a non-aqueous solvent.

Upon application of the negative pressure to the channel 140, the manifold member 150 may collapse, which may cause the skin 24 around the wound 22 to stretch. This may dilate vessels/lymphatics and allow improved fluid movement to the wound 22. In some embodiments, the manifold member 150 may collapse substantially along the width axis 155 (shown in FIG. 2B) of the manifold member 150 upon application of the negative pressure to the channel 140, thereby increasing the effect of vessel dilation.

Application of the negative pressure to the channel 140 may further result in application of the negative pressure to the periwound, which contains critical vasculature that supplies the wound 22 with elements needed for healing, such as improved perfusion, oxygenation, and stress-induced cell proliferation.

Moreover, as the negative pressure may only be applied to the channel 140, the negative pressure source 12 of a cheaper and less powerful variety may be used in the wound therapy system 10, as compared to conventional negative pressure systems. Advantageously, the channel 140 may additionally function as a protective border and absorb loads from accidental bumps, thereby providing protection to the wound 22.

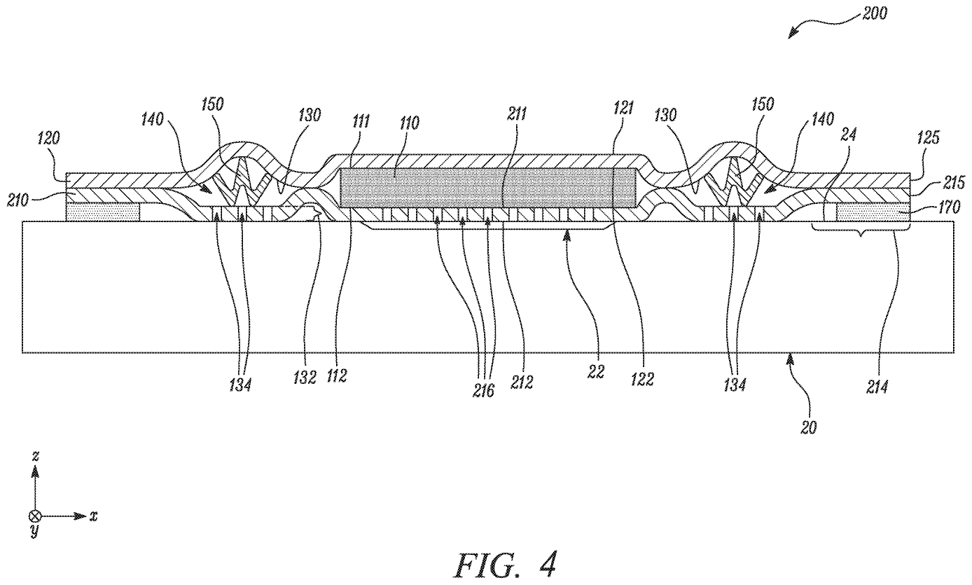

FIG. 4 illustrates a schematic cross-sectional view of a wound dressing 200 according to an embodiment of the present disclosure. The wound dressing 200 may be used alternatively to the wound dressing 100 in the wound therapy system 10 of FIG. 1.

The wound dressing 200 is substantially similar to the wound dressing 100 of FIG. 2A, with like elements designated by like reference characters. However, the wound dressing 200 includes additional components as compared to the wound dressing 100.

Specifically, the wound dressing 200 may further include an interface layer 210. The interface layer 210 may include a first interface surface 211 and a second interface surface 212 opposite to the first interface surface 211. The first interface surface 211 may be sealed to the second cover surface 122, such that the cover layer 120 and the interface layer 210 together form the channel wall 130. The first interface surface 211 may be sealed to the second cover surface 122 by any suitable method, such as adhesive lamination, heat lamination, and the like. Furthermore, in the illustrated embodiment of FIG. 4, the interface layer 210 forms the bottom portion 132 of the channel wall 130. As discussed above, in some embodiments, the bottom portion 132 further includes an adhesive configured to adhere the bottom portion 132 to the skin 24. In some embodiments, the bottom portion 132 includes both the adhesive and the plurality of through openings 134.

The interface layer 210 may further include an interface perimeter 215 disposed between the first interface surface 211 and the second interface surface 212. The second interface surface 212 may include an outer portion 214 disposed between the interface perimeter 215 and the channel 140. The wound dressing 200 may further include the adhesive layer 170 at least partially disposed on the outer portion 214 and configured to removably connect the wound dressing 200 to the skin 24 around the wound 22. The interface layer 210 may be made, for example, from a polymer film (e.g., a polyurethane film) or a medical textile (e.g., Asahi nylon).

In some embodiments, the interface layer 210 may be non-coextensive with the cover layer 120. Specifically, the interface layer 210 may be only attached to a portion of the cover layer 120 to form a portion of the channel wall 130, such that upon removable connection of the wound dressing 200 to the skin 24 around the wound 22, the absorbent pad 110 can directly engage with the wound 22.

In the illustrated embodiment of FIG. 4, the interface layer 210 is coextensive with the cover layer 120. In such embodiments, the absorbent pad 110 may be disposed between the cover layer 120 and the interface layer 210. In other words, the absorbent pad 110 may be “sandwiched” between the cover layer 120 and the interface layer 210. Specifically, the absorbent pad 110 may be disposed between the second cover surface 122 and the first interface surface 211.

The interface layer 210 may further include a plurality of through openings 216 configured to fluidly couple the absorbent pad 110 to the wound 22, such that the absorbent pad 110 receives exudate from the wound 22 via the plurality of through openings 216. The plurality of through openings 216 may be adjacent to the absorbent pad 110.

The wound therapy system 10 and the wound dressings 100, 200 may promote perfusion at the wound site 20 and promote healing of the wound 22. Specifically, during use of the wound therapy system 10 and the wound dressings 100, 200, the negative pressure applied to the channel 140 may allow the periwound to be stretched, thereby dilating vessels/lymphatics, and allowing improved fluid movement.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

1. A wound dressing for a wound, the wound dressing comprising:
an absorbent pad comprising a first pad surface and a second pad surface opposite to the first pad surface, wherein the second pad surface is configured to face the wound;
a cover layer comprising a first cover surface and a second cover surface opposite to the first cover surface, wherein the second cover surface is attached to and faces the first pad surface, and wherein the cover layer comprises at least a portion of a channel wall;
a channel defined by the channel wall, wherein the channel surrounds the absorbent pad and is spaced apart from the absorbent pad, wherein the channel is continuous and fluidly isolated from the absorbent pad, and wherein the channel is configured to receive a negative pressure; and
a manifold member disposed within the channel and at least partially engaging the channel wall, wherein the manifold member is collapsible in response to the negative pressure applied to the channel.

2. The wound dressing of claim 1, further comprising a port fluidly coupled to the channel, and wherein the channel is configured to receive the negative pressure via the port.

3. The wound dressing of claim 1, wherein only the channel of the wound dressing is configured to receive the negative pressure, and wherein the absorbent pad is not configured to receive any negative pressure.

4. The wound dressing of claim 1, wherein the manifold member comprises a cross-sectional plane, a width axis disposed in the cross-sectional plane along a width of the manifold member, and a height axis perpendicular to the width axis, and wherein the manifold member is collapsible substantially along the width axis upon application of the negative pressure to the channel.

5. The wound dressing of claim 4, wherein the manifold member comprises a maximum width along the width axis, and wherein the maximum width of the manifold member decreases upon application of the negative pressure to the channel.

6. (canceled)

7. The wound dressing of claim 1, wherein the manifold member comprises a plurality of vertices, and wherein each of the plurality of vertices engages the channel wall.

8. The wound dressing of claim 1, wherein the cover layer is sealed to itself to form the channel wall.

9. The wound dressing of claim 1, wherein the cover layer further comprises a cover perimeter extending between the first cover surface and the second cover surface, wherein the second cover surface of the cover layer comprises an outer portion disposed between the cover perimeter and the channel, and wherein the wound dressing further comprises an adhesive layer at least partially disposed on the outer portion and configured to removably connect the wound dressing to a skin around the wound.

10. The wound dressing of claim 9, wherein an area of the second cover surface is greater than an area of the first pad surface, such that the cover perimeter surrounds the absorbent pad.

11. The wound dressing of claim 1, further comprising an interface layer comprising a first interface surface sealed to the second cover surface, such that the cover layer and the interface layer together form the channel wall.

12. The wound dressing of claim 11, wherein the interface layer further comprises a second interface surface opposite to the first interface surface, and an interface perimeter disposed between the first interface surface and the second interface surface, wherein the second interface surface comprises an outer portion disposed between the interface perimeter and the channel, and wherein the wound dressing further comprises an adhesive layer at least partially disposed on the outer portion and configured to removably connect the wound dressing to a skin around the wound.

13. The wound dressing of claim 11, wherein the absorbent pad is disposed between the cover layer and the interface layer.

14. The wound dressing of claim 13, wherein the interface layer comprises a plurality of through openings configured to fluidly couple the absorbent pad to the wound, such that the absorbent pad is configured to receive exudate from the wound via the plurality of through openings.

15. The wound dressing of claim 1, wherein the manifold member comprises a foam.

16. The wound dressing of claim 1, wherein the channel wall comprises a bottom portion configured to face a skin around the wound, and wherein the bottom portion comprises a plurality of through openings.

17. A wound therapy system for a wound, the wound therapy system comprising:
a wound dressing comprising:
an absorbent pad comprising a first pad surface and a second pad surface opposite to the first pad surface, wherein the second pad surface is configured to face the wound;
a cover layer comprising a first cover surface and a second cover surface opposite to the first cover surface, wherein the second cover surface is attached to and faces the first pad surface, and wherein the cover layer comprises at least a portion of a channel wall;
a channel defined by the channel wall, wherein the channel surrounds the absorbent pad and is spaced apart from the absorbent pad, and wherein the channel is continuous and fluidly isolated from the absorbent pad; and
a manifold member disposed within the channel and at least partially engaging the channel wall; and
a negative pressure source configured to fluidly communicate with the channel and to apply a negative pressure to the channel;
wherein the manifold member is configured to collapse in response to the negative pressure applied to the channel.

18. (canceled)

19. The wound therapy system of claim 17, wherein the negative pressure source is configured to be fluidly coupled to only the channel of the wound dressing.

20. The wound therapy system of claim 17, wherein the manifold member is configured to collapse substantially along a width of the manifold upon application of the negative pressure to the channel.

21-23. (canceled)

24. The wound therapy system of claim 17, wherein the cover layer is sealed to itself to form the channel wall.

25. (canceled)

26. (canceled)

27. The wound therapy system of claim 17, further comprising an interface layer comprising a first interface surface sealed to the second cover surface, such that the cover layer and the interface layer together form the channel wall.

28. (canceled)

29. The wound therapy system of claim 27, wherein the absorbent pad is disposed between the cover layer and the interface layer.

30. The wound therapy system of claim 29, wherein the interface layer comprises a plurality of through openings configured to fluidly couple the absorbent pad to the wound, such that the absorbent pad is configured to receive exudate from the wound via the plurality of through openings.

31. (canceled)

32. (canceled)
